(19) United States
(12) Patent Application Publication
(10) Pub. No.: US 2026/0000888 A1
(43) Pub. Date: Jan. 1, 2026

(54) Apparatus And Method For Array Positioning

(71) Applicant: NOVOCURE GMBH, Baar (CH)

(72) Inventors: Niven Curtis-Woodcock, Cambridge (UA); Henry James Moorman, Cambridge (UA); Michael Robert George Jones, Cambridge (UA); Andrew Duncan Pidgeon, Cambridge (UA)

(21) Appl. No.: 19/252,086

(22) Filed: Jun. 27, 2025

Related U.S. Application Data
(60) Provisional application No. 63/665,370, filed on Jun. 28, 2024.

(51) Int. Cl. A61N 1/36; A61N 1/04

(52) U.S. Cl. CPC A61N 1/36002; A61N 1/0476; A61N 1/0492

(57) ABSTRACT

An apparatus for positioning at least one electrode array on a back of a user. The apparatus can include a body that is foldable about and between a first configuration and a second, expanded configuration. The body can include a seat portion and a back portion. In the second, expanded configuration, the seat portion of the body can be configured for positioning on a seat of a chair and the back portion can be configured to rest against a back of the chair. The back portion of the body can have a forward-facing surface comprising at least one marking or attachment element, the at least one marking or attachment element indicating a location for placement of the at least one electrode array.

### CROSS-REFERENCE TO RELATED APPLICATIONS

This application claims priority to U.S. Provisional Patent Application No. 63/665,370, filed June 28, 2024, the entirety of which is hereby incorporated by reference herein.

### FIELD

This disclosure relates to systems and methods for positioning electrode arrays on a user.

### BACKGROUND

Tumor Treating Fields (TTFields) therapy is a proven approach for treating tumors using alternating electric fields at frequencies between 50 kHz - 1 MHz, more commonly, 100-500 kHz. In current commercial systems, the alternating electric fields are induced by electrode assemblies (e.g., arrays of capacitively coupled electrodes, also called transducer arrays or electrode arrays) placed on opposite sides of a target region of the subject's body. When an AC voltage is applied between opposing electrode arrays, an AC current is coupled through the electrode arrays and into the subject's body.

Proper positioning of electrode arrays relative to each other and a target region (e.g., a tumor) can affect performance of treatment. However, proper placement can be difficult, particularly when the subject is placing the electrode arrays on himself/herself. Thus, this difficulty can diminish the independence of the subject, requiring the subject to have another person (helper) position the electrode arrays. Accordingly, a way to assist a subject with properly positioning one or more electrode arrays is desirable.

### SUMMARY

TTFields are approved for the treatment of glioblastoma multiforme (GBM), and may be delivered, for example, via the OPTUNE* system (Novocure GmbH, Baar, Switzerland), which includes transducer arrays placed on the user's shaved head. More recently, TTFields therapy has been approved as a combination therapy with chemotherapy for malignant pleural mesothelioma (MPM) and non-small cell lung cancer (NSCLC), and may find use in treating tumors in other parts of the body. For example, recent studies have shown that TTFields treatment can reduce progression of metastases to the brain from non- small cell lung cancer (NSCLC).

Disclosed herein, in one aspect, an apparatus for positioning at least one electrode array on an individual (a user). For example, the apparatus can be used to position the at least one electrode array on a torso of the user (e.g., a back of the user). The apparatus can include a body that is foldable about and between a first configuration and a second, expanded configuration. The body can include a seat portion and a back portion. In the second, expanded configuration, the seat portion of the body can be configured for positioning on a seat of a chair and the back portion can be configured to rest against a back of the chair. The back portion of the body can have a forward-facing surface comprising at least one marking or attachment element, the at least one marking or attachment element indicating a location for placement of the at least one electrode array.

Systems, methods, and kits using the apparatus are also disclosed herein.

### BRIEF DESCRIPTION OF THE DRAWINGS

FIG. 1 illustrates an apparatus being used for positioning at least one electrode array on a user.

FIG. 2 illustrates the apparatus of FIG. 1 in a first (folded, compact) configuration.

FIG. 3 illustrates the apparatus of FIG. 1 in a partially expanded position between the first configuration and a second, expanded configuration.

FIG. 4 illustrates the apparatus of FIG. 1 in the second, expanded configuration.

FIG. 5 illustrates a map showing placement locations of electrode arrays relative to markings or attachment element(s) on a forward-facing surface of the back portion of the body of an exemplary positioning of at least one electrode array on a user.

FIG. 6 illustrates placement of an electrode array on the apparatus.

FIG. 7 illustrates placement of the apparatus on a chair.

FIG. 8 illustrates removal of a release liner of an electrode array.

FIG. 9 illustrates a user sitting against the apparatus for attachment of the electrode arrays to the user.

FIG. 10 illustrates the user moving from the apparatus with the electrode arrays attached to the user in desired locations.

Various embodiments are described in detail below with reference to the accompanying drawings, wherein like reference numerals represent like elements, and wherein descriptions of like elements may not be repeated for every embodiment, but may be considered to be the same if previously described herein.

### DESCRIPTION OF THE PREFERRED EMBODIMENTS

As discussed above, proper placement of electrode arrays can be difficult, particularly when the subject (a user) is placing the electrode arrays on himself/herself. This application relates to accurately positioning electrode arrays that may be used, e.g., for delivering TTFields to a subject's body (for example, the lungs) and treating one or more cancers or tumors located in the subject's body (e.g., the lungs). The subject may then reproduce the accurate positioning of the electrode arrays for future application of the arrays on the back of the subject, for example, by appropriately marking the apparatus.

The present invention can be understood more readily by reference to the following detailed description, examples, drawings, and claims, and their previous and following description. However, it is to be understood that this invention is not limited to the specific apparatuses, devices, systems, and/or methods disclosed unless otherwise specified, and as such, of course, can vary.

Headings are provided for convenience only and are not to be construed to limit the invention in any manner. Embodiments illustrated under any heading or in any portion of the disclosure may be combined with embodiments illustrated under the same or any other heading or other portion of the disclosure.

Any combination of the elements described herein in all possible variations thereof is encompassed by the invention unless otherwise indicated herein or otherwise clearly contradicted by context.

As used in the specification and the appended claims, the singular forms "a," "an," and "the" include plural referents unless the context clearly dictates otherwise. Thus, it is contemplated that disclosure of a singular form of an element can provide support for embodiments in which only a single such element is provided, as well as support for embodiments in which a plurality of such elements are provided.

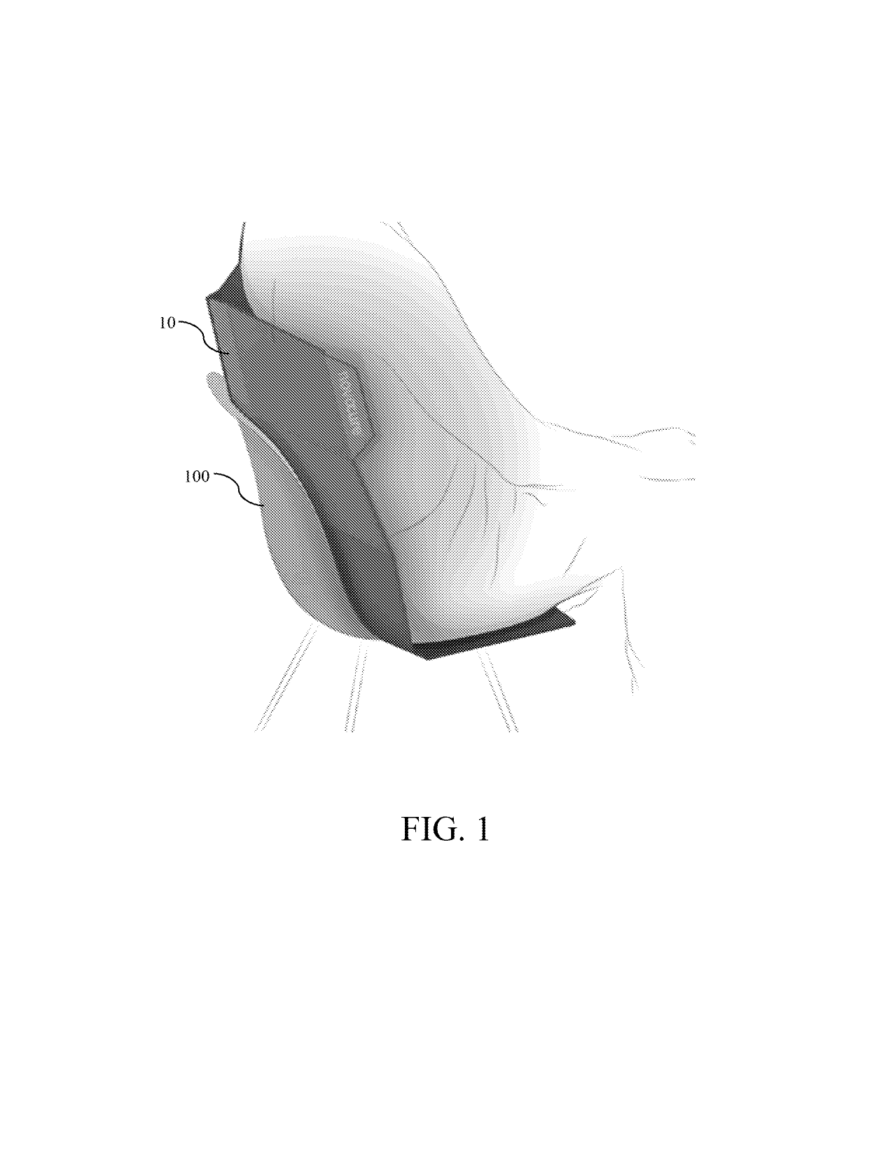

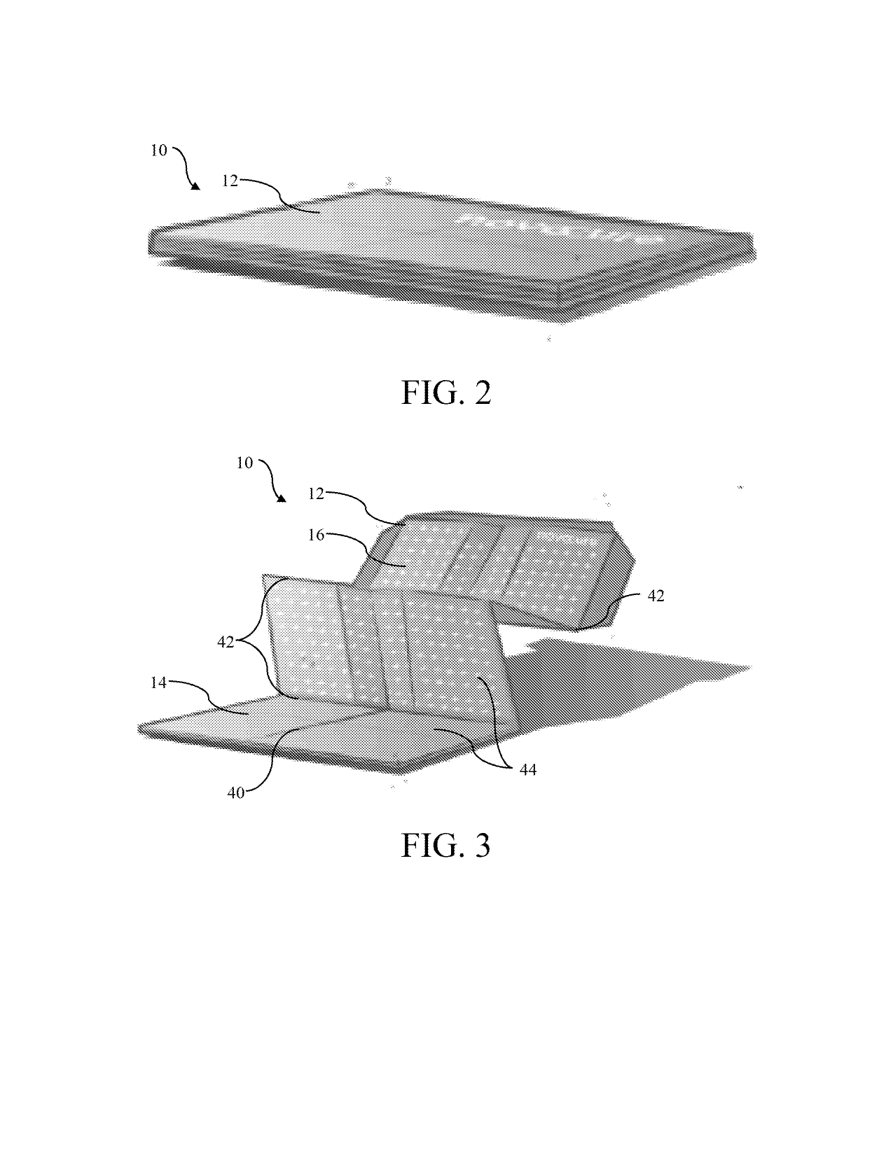

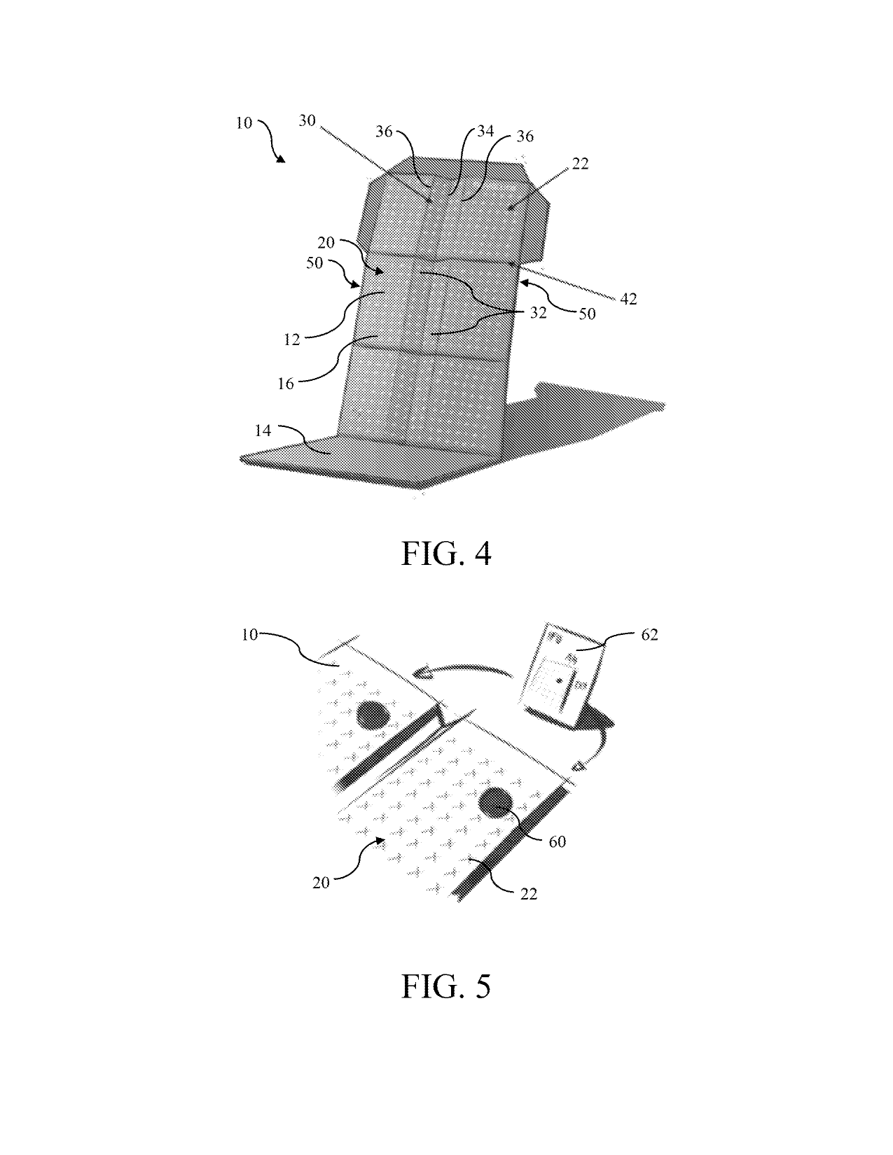

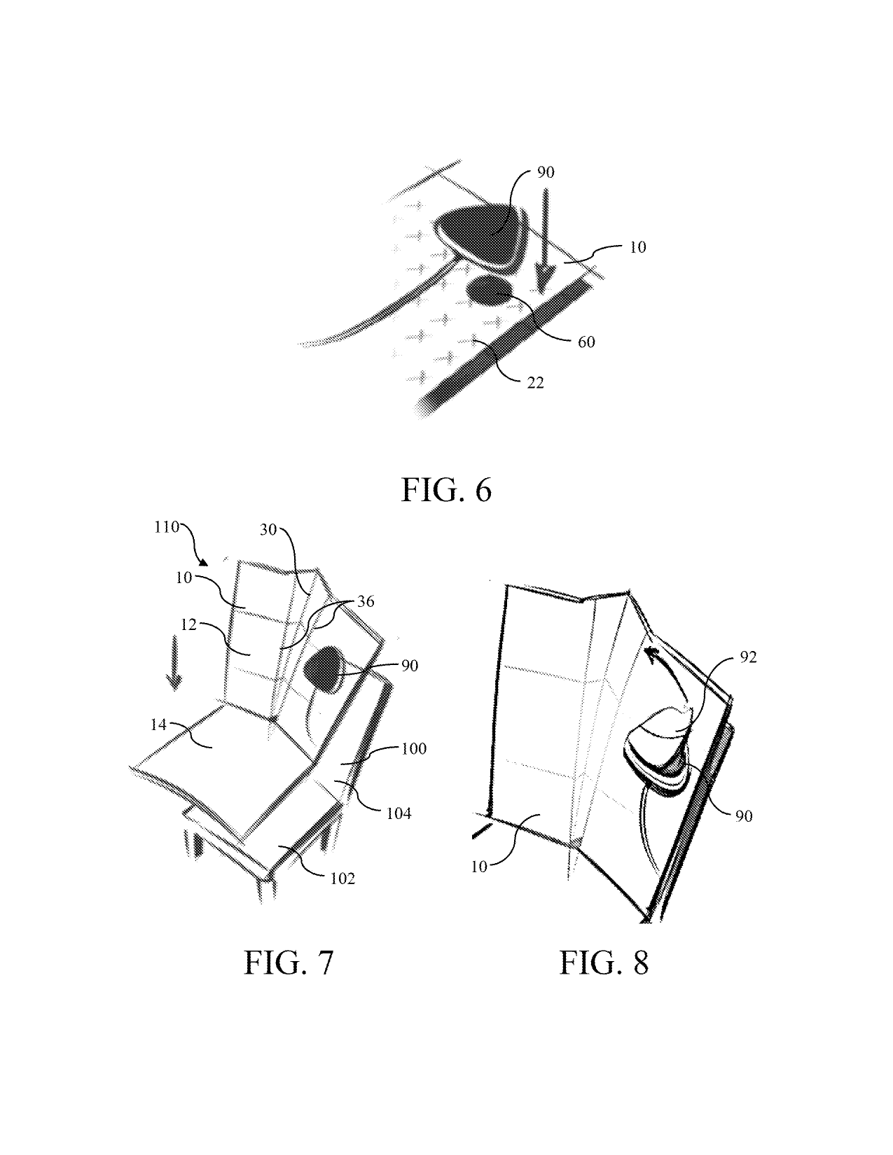

FIG.1 illustrates an apparatus 10 being used for positioning at least one electrode array 90 (FIG. 6) on a user. FIGS. 2-4 illustrate the apparatus in, respectively: a first (folded, compact) configuration (FIG. 2); a partially expanded configuration (FIG. 3) between the first configuration and a second, expanded configuration; and the second, expanded configuration (FIG. 4).

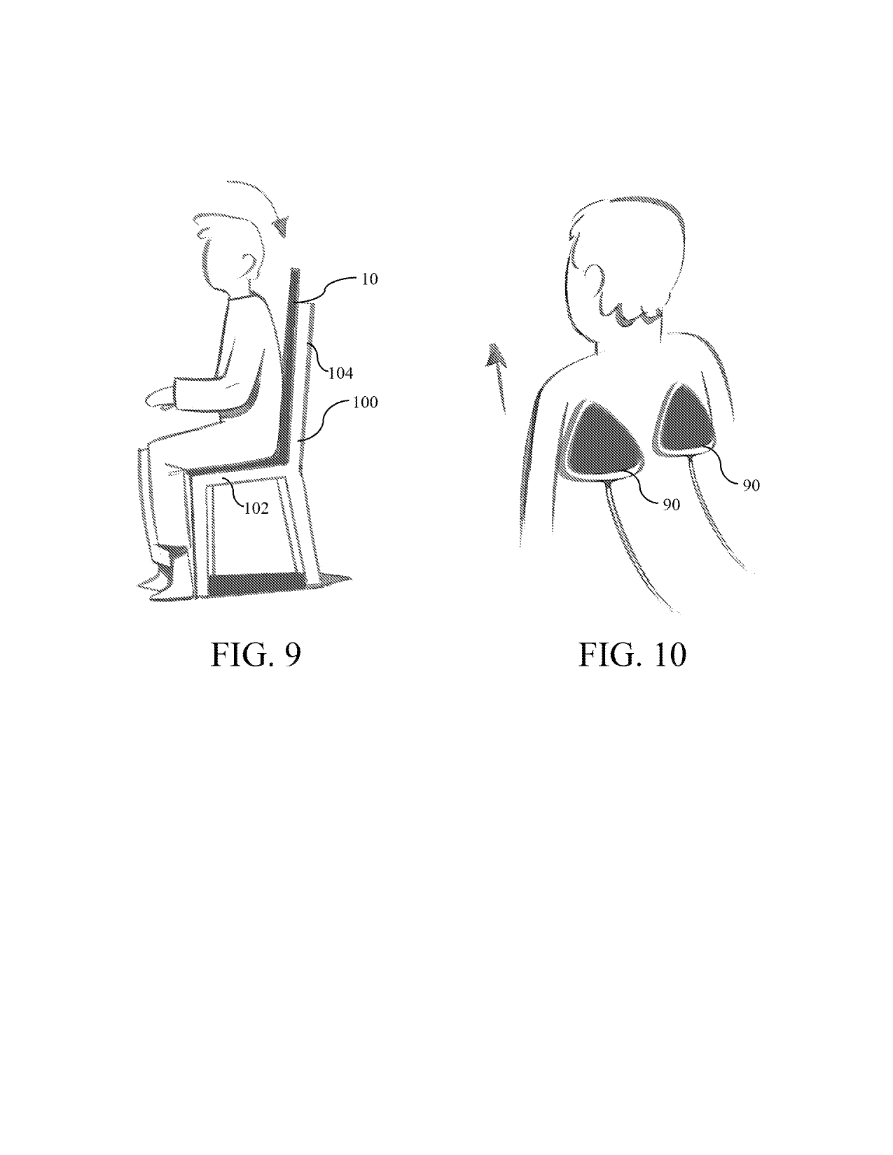

Referring to FIGS. 1-4, the apparatus 10 can include a body 12 that is foldable about and between the first configuration (FIG. 2) and the second, expanded configuration (FIG. 4). The body 12 can have a seat portion 14 and a back portion 16. In the second, expanded configuration, the seat portion 14 of the body 12 can be configured for positioning on a seat 102 (FIGS. 7, 9) of a chair 100, and the back portion 16 can be configured to rest against a back 104 (FIGS. 7, 9) of the chair 100. The back portion 16 of the body 12 can have a forward-facing surface 20 comprising at least one marking 22 or attachment element 60 (FIG. 5). The at least one marking 22 or attachment element 60 can be used as a guide to indicate a location for placement of the at least one electrode array 90 (FIGS. 6-8, 10).

In some aspects, the at least one marking 22 can comprise a grid or a pattern representing a grid. Optionally, in these aspects, the user can be provided with additional information for positioning the at least one electrode array 90 relative to the grid. For example, the additional information can include written instructions (e.g., grid coordinates, and/or an explanation of one or more steps for using the apparatus 10) and/or an illustration indicating the location for placement of each electrode array of the at least one electrode array.

In other aspects, the at least one marking 22 can be specific to the user. For example, the at least one marking 22 can include a user-specific marking printed on the body 12 of the apparatus 10 for indicating a location for the at least one electrode array 90. Optionally, in these aspects, the at least one marking 22 can correspond to a center of an electrode array 90 or an outer perimeter (or portion thereof) of an electrode array. For example, the at least one marking 22 can comprise a marking tracing a portion of or an entirety of an outer perimeter of each electrode array of the at least one electrode array 90. As another example, the marking 22 can comprise a dot, an X, an arrow, or another marking that indicates a position for the geometric center (e.g., centroid) of the at least one electrode array 90. Alternatively, one or more pre-positioned attachment elements 60 on the body 12 of the apparatus 10 can indicate a position for the at least one electrode array 90. The attachment element(s) 60 can releasably attach an electrode array to the body 12.

In some aspects, the body 12 can comprise paper. In further aspects, the body 12 can comprise cardboard (e.g., optionally, corrugated cardboard). In other aspects, the body 12 can comprise a flexible polymeric sheet.

The body 12 has a weight. In some aspects, the body 12 can be sufficiently rigid to stay in the expanded configuration under the weight of the body when resting in a chair 100 (FIG. 9). That is, the body 12 can be sufficiently rigid to avoid collapsing when placed in a chair.

In some aspects, when the body 12 is in the expanded configuration, the back portion 16 can have an alignment fold 30 (FIG. 4). The alignment fold 30 can be a longitudinal fold that permits tactile indication of a position of the body 12 relative to the user. For example, the alignment fold 30 can be configured to be aligned with the spine of the user along a transverse axis. In some aspects, the alignment fold 30 can comprise a pair of central portions 32 that each extend between a central fold 34 and a respective fold 36 spaced laterally from the central fold. The pair of central portions can extend rearwardly from the respective folds 36 to the central fold 34. In this way, the alignment fold 30 can define a V-shaped recess along the forward-facing surface 20 of the back portion 16 of the body 12. In some aspects, and as illustrated in FIG. 7, the respective folds 36 can meet at, or converge toward, the seat portion 14. In other aspects, the respective folds 36 can be generally parallel (e.g., parallel or within 10 degrees of being parallel).

The body 12 can comprise at least one longitudinally extending fold 40 (FIG.3) and at least one transversely extending fold 42 (FIG. 3). Optionally, the at least one longitudinally extending fold 40 can comprise the center fold 34 of the alignment fold 30. In some aspects, body 12 can comprise at least two, or at least three, transversely extending folds 42. In various embodiments, the body 12 can comprise at least eight segments 44, wherein adjacent segments meet at respective folds (FIG. 3). For example, referring to FIG. 3, the seat portion 14 of the body 12 can comprise two segments 44, and the back portion 16 can comprise six segments 44. Accordingly, in these optional aspects, when folded, the body 12 can have a footprint that is about 1/8 of a footprint of the body 12 once unfolded and lying flat. In various further aspects, the body 12 can have a footprint that is less than 33% of, or less than 28% of, or less than 25% of, or less than 20% of, or less than 15% of the footprint of the body 12 once unfolded and lying flat.

The body 12 can have opposed longitudinally extending edges 50 (FIG. 4). In some aspects, the longitudinally extending edges 50 can be parallel or generally parallel (e.g., within 10 degrees of being parallel). In other aspects, as shown in FIG. 7, the longitudinally extending edges 50 can diverge in a direction moving upwardly along the back portion 16 of the body 12 from the seat portion 14.

Referring to FIG. 5, the apparatus 10 can comprise at least one attachment element 60 on the forward-facing surface 20 of the back portion 16 of the body 12. For example, in some aspects, the at least one attachment element 60 can comprise a hook and/or loop fastener surface (formed by, for example, Velcro* hook and loop fastener provided by Velcro Companies of Manchester, NH) or an adhesive. The attachment element(s) 60 can releasably attach an electrode array to the body 12. Optionally, the apparatus 10 may be accompanied by instructions 62, such as, for example, an instruction card, a manual, a booklet, electronic media, or the like. The instructions 62 can convey to the user how to use the apparatus 10 (for example, including a placement map/diagram).

FIGS. 5-10 illustrate exemplary method steps for using the apparatus 10. As should be understood, the steps can be performed in any logical order and are not limited to the order shown. After the body 12 has been opened to the second, expanded configuration, at least one electrode array 90 can be coupled to the forward-facing surface 20 of the back portion 16 of the body.

For example, as illustrated in FIG. 5, attachment element(s) 60 can be placed on the forward-facing surface 20 of the back portion 16 of the body based on the instructions 62. The instructions 62 can include grid coordinates and/or an illustration. In one aspect, the attachment element(s) 60 can comprise adhesive on one side and hook or loop fastener on the other. Referring to FIG. 6, one or more electrode arrays 90 can be coupled to the body via the attachment element(s) 60. Referring to FIG. 7, the apparatus 10 can be placed in a chair 100, with the seat portion 14 of the body 12 resting on the seat 102 of the chair, and the back portion 16 resting on the back 104 of the chair. Referring to FIG. 8, a release liner 92 of each electrode array 90 can be removed from the electrode array to expose adhesive of the electrode array. As shown in FIG. 9, a user can sit against the apparatus 10. For example, the user can use the alignment fold 30 to locate her spine relative to the apparatus 10. Optionally, the user can first sit in the chair 100 and then lean back to press her torso against the apparatus 10. The user can then move away from the apparatus. The attachment element(s) 60 can have a lesser holding force than the adhesive of the electrode array 90 so that the electrode arrays 90 remain attached to the user (FIG. 10). Optionally, the user can sit against the apparatus 10 for a predetermined time to ensure that the adhesive has sufficiently attached to the user.

A system 110 (FIG. 7) can comprise an apparatus 10 and one or more electrode arrays 90 coupled to the forward-facing surface 20 of the back portion 16 of the body 12 of the apparatus. Optionally, in these aspects, the at least one marking 22 or placement of an attachment element can be customized for a particular user.

A kit can comprise an apparatus 10. In further aspects, the kit can comprise one or a plurality of electrode arrays 90. In still further aspects, the kit can comprise instructions 62 designating the location for placement of the at least one electrode array 90 relative to the at least one marking 22. In further aspects, the kit can comprise at least one attachment element 60 that is configured to couple to the forward-facing surface 20 of the back portion 16 of the body. Optionally, said instructions 62 can comprise instructions for locating the at least one attachment element 60 on the apparatus 10 (e.g., relative to, or instead of, the at least one marking 22).

### EXEMPLARY ASPECTS

In view of the described products, systems, and methods and variations thereof, herein below are described certain more particularly described aspects of the invention. These particularly recited aspects should not however be interpreted to have any limiting effect on any different claims containing different or more general teachings described herein, or that the "particular" aspects are somehow limited in some way other than the inherent meanings of the language literally used therein.

Aspect 1: An apparatus for positioning at least one electrode array on a user, the apparatus comprising: a body that is foldable about and between a first configuration and a second, expanded configuration, wherein the body comprises a seat portion and a back portion, wherein in the second, expanded configuration, the seat portion of the body is configured for positioning on a seat of a chair and the back portion is configured to rest against a back of the chair, wherein the back portion of the body has a forward-facing surface comprising at least one marking or attachment element, the at least one marking or attachment element indicating a location for placement of the at least one electrode array.

Aspect 2: The apparatus of aspect 1, wherein the body comprises paper.

Aspect 3: The apparatus of aspect 1 or aspect 2, wherein the body comprises cardboard or corrugated cardboard.

Aspect 4: The apparatus of any one of the preceding aspects, wherein the body comprises a flexible polymeric sheet.

Aspect 5: The apparatus of any one of the preceding aspects, wherein the body has a weight, wherein the body is sufficiently rigid to stay in the expanded configuration under the weight of the body when resting in a chair.

Aspect 6: The apparatus of any one of the preceding aspects, wherein the forward- facing surface of the back portion of the body comprises at least one marking, and the at least one marking comprises a grid or a pattern representing a grid.

Aspect 7: The apparatus of any one of the preceding aspects, wherein, in the expanded configuration, the back portion comprises an alignment fold.

Aspect 8: The apparatus of aspect 7, wherein the alignment fold comprises a pair of central portions that each extend between a central fold and a respective fold spaced laterally from the central fold, wherein the pair of central portions extend rearwardly from the respective folds to the central fold.

Aspect 9: The apparatus of aspect 8, wherein the respective folds of the central portions meet at the seat portion.

Aspect 10: The apparatus of any one of the preceding aspects, wherein the body comprises at least one longitudinally extending fold and at least one transversely extending fold.

Aspect 11: The apparatus of aspect 10, wherein the at least one transversely extending fold comprises at least three transversely extending folds.

Aspect 12: The apparatus of any one of the preceding aspects, wherein the body comprises at least eight segments, wherein adjacent segments of the at least eight segments meet at respective folds.

Aspect 13: The apparatus of any one of the preceding aspects, wherein the body comprises opposed longitudinally extending edges, wherein the longitudinally extending edges are generally parallel.

Aspect 14: The apparatus of any one of the preceding aspects, comprising at least one attachment element on the forward-facing surface of the back portion of the body.

Aspect 15: The apparatus of aspect 14, wherein the at least one attachment element comprises a hook and/or loop fastener surface or an adhesive.

Aspect 16: A method of using the apparatus of any one of the preceding aspects, the method comprising: coupling at least one electrode array to the forward-facing surface of the back portion of the body.

Aspect 17: The method of aspect 16, further comprising: positioning the body of the apparatus on a chair; and sitting on the seat portion of the body of the apparatus supported by the chair, wherein a torso of the user is positioned against the at [east one electrode array so that the at least one electrode array adheres to the torso of the user.

Aspect 18: The method of aspect 17, further comprising moving forwardly away from the back portion of the body to detach the at least one electrode array from the forward- facing surface of the back portion of the body.

Aspect 19: A system comprising: an apparatus as in any one of the preceding aspects; and one or more electrode arrays coupled to the forward-facing surface of the back portion of the body of the apparatus.

Aspect 20: The system of aspect 19, wherein a location of the at least one marking or attachment element is customized for a particular user.

Aspect 21: A kit comprising: an apparatus as in any one of the preceding aspects; and a plurality of electrode arrays.

Aspect 22: The kit of aspect 21, further comprising instructions designating the location for placement of the at least one electrode array relative to the at least one marking or attachment element.

Aspect 23: The kit of aspect 21 or aspect 22, further comprising at least one attachment element that is configured to couple to the forward-facing surface of the back portion of the body.

Aspect 24: The kit of any one of aspects 21-23, wherein placement of the at least one marking or attachment element is customized for a particular user.

Aspect 25: A method for positioning at least one electrode array on a user using an apparatus comprising a body that is foldable about and between a first configuration and a second, expanded configuration, wherein the body comprises a seat portion and a back portion, wherein in the second, expanded configuration, the seat portion of the body is configured for positioning on a seat of a chair and the back portion is configured to rest against a back of the chair, the method comprising: positioning at least one electrode array relative to at least one marking or attachment element on a forward-facing surface of the back portion of the body, wherein the at least one marking or attachment element is indicative of a location for placement of the at least one electrode array.

Aspect 26: The method of aspect 25, further comprising sitting on the apparatus with the seat portion of the body positioned on the seat of the chair and the back portion of the body positioned against the back portion of the chair, wherein a torso of the user is positioned against the at least one electrode array so that the at least one electrode array adheres to the torso of the user.

Aspect 27: The method of aspect 25 or aspect 26, further comprising: opening the body of the apparatus from the first configuration to the second configuration; and positioning the seat portion of the body on the seat of the chair and the back portion of the body on the back portion of the chair.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An apparatus for positioning at least one electrode array on a user, the apparatus comprising:
a body that is foldable about and between a first configuration and a second, expanded configuration, wherein the body comprises a seat portion and a back portion, wherein in the second, expanded configuration, the seat portion of the body is configured for positioning on a seat of a chair and the back portion is configured to rest against a back of the chair,
wherein the back portion of the body has a forward-facing surface comprising at least one marking or attachment element, the at least one marking or attachment element indicating a location for placement of the at least one electrode array.

2. The apparatus of claim 1, wherein the body comprises paper, cardboard, or corrugated cardboard.

3. The apparatus of claim 1, wherein the body comprises a flexible polymeric sheet.

4. The apparatus of claim 1, wherein the body has a weight, wherein the body is sufficiently rigid to stay in the expanded configuration under the weight of the body when resting in a chair.

5. The apparatus of claim 1, wherein the forward-facing surface of the back portion of the body comprises at least one marking, and the at least one marking comprises a grid or a pattern representing a grid.

6. The apparatus of claim 1, wherein, in the expanded configuration, the back portion comprises an alignment fold.

7. The apparatus of claim 6, wherein the alignment fold comprises a pair of central portions that each extend between a central fold and a respective fold spaced laterally from the central fold, wherein the pair of central portions extend rearwardly from the respective folds to the central fold.

8. The apparatus of claim 1, wherein the body comprises at least one longitudinally extending fold and at least one transversely extending fold.

9. The apparatus of claim 8, wherein the at least one transversely extending fold comprises at least three transversely extending folds.

10. The apparatus of claim 1, wherein the body comprises at least eight segments, wherein adjacent segments of the at least eight segments meet at respective folds.

11. The apparatus of claim 1, wherein the body comprises opposed longitudinally extending edges, wherein the longitudinally extending edges are generally parallel.

12. The apparatus of claim 1, comprising at least one attachment element on the forward- facing surface of the back portion of the body.

13. The apparatus of claim 12, wherein the at least one attachment element comprises a hook and/or loop fastener surface or an adhesive.

14. A kit comprising:
an apparatus comprising:
a body that is foldable about and between a first configuration and a second, expanded configuration, wherein the body comprises a seat portion and a back portion, wherein in the second, expanded configuration, the seat portion of the body is configured for positioning on a seat of a chair and the back portion is configured to rest against a back of the chair,
wherein the back portion of the body has a forward-facing surface comprising at least one marking or attachment element, the at least one marking or attachment element indicating a location for placement of at least one electrode array; and
a plurality of electrode arrays.

15. The kit of claim 14 further comprising instructions designating the location for placement of the at least one electrode array relative to the at least one marking or attachment element.

16. The kit of claim 14, further comprising at least one attachment element that is configured to couple to the forward-facing surface of the back portion of the body.

17. The kit of claim 14, wherein placement of the at least one marking or attachment element is customized for a particular user.

18. A method for positioning at least one electrode array on a user using an apparatus comprising a body that is foldable about and between a first configuration and a second, expanded configuration, wherein the body comprises a seat portion and a back portion, wherein in the second, expanded configuration, the seat portion of the body is configured for positioning on a seat of a chair and the back portion is configured to rest against a back of the chair, the method comprising:
positioning at least one electrode array relative to at least one marking or attachment element on a forward-facing surface of the back portion of the body, wherein the at least one marking or attachment element is indicative of a location for placement of the at least one electrode array.

19. The method of claim 18, further comprising sitting on the apparatus with the seat portion of the body positioned on the seat of the chair and the back portion of the body positioned against the back portion of the chair, wherein a torso of the user is positioned against the at least one electrode array so that the at least one electrode array adheres to the torso of the user.

20. The method of claim 18, further comprising:
opening the body of the apparatus from the first configuration to the second configuration; and
positioning the seat portion of the body on the seat of the chair and the back portion of the body against the back portion of the chair.
